(19) United States
(12) Patent Application Publication
(10) Pub. No.: US 2009/0112222 A1
(43) Pub. Date: Apr. 30, 2009

(54) IOL Insertion Apparatus

(75) Inventors: Daniel P. Barrows, Rochester, NY (US); Edward A. Vaquero, Fairport, NY (US)

Correspondence Address: Bausch & Lomb Incorporated, One Bausch & Lomb Place, Rochester, NY, 14604-2701, US

(73) Assignee: BAUSCH & LOMB INCORPORATED, Rochester, NY (US)

(21) Appl. No.: 11/924,376

(22) Filed: Oct. 25, 2007

(51) Int. Cl. A61F 9/00

(52) U.S. Cl. 606/107

(57) ABSTRACT

An apparatus for inserting an intraocular lens through a small incision into an eye is disclosed comprising a hollow tube including an interior wall defining a hollow space through which an intraocular lens may be passed and an outlet through which the intraocular lens may be passed from the hollow space into the eye, wherein at least the hollow tube of the apparatus is obtained from at least a polymeric resin comprising a polymer backbone and one or more pendent groups having peroxide functionality and covalently linked to the polymer backbone.

### BACKGROUND OF THE INVENTION

1. Technical Field

The present invention generally relates to an improved apparatus for inserting an intraocular lens through a small incision into an eye. More particularly, the present invention generally relates to an apparatus which has enhanced lubricity and is useful for inserting a foldable intraocular lens into an eye, to methods for making such apparatus and to methods using such apparatus to insert a foldable intraocular lens into an eye.

2. Description of the Related Art

An intraocular lens (IOL) is implanted in the eye, for example, as a replacement for the natural crystalline lens after cataract surgery or to alter the optical properties of an eye in which the natural lens remains, e.g., provide vision, correct vision, etc). IOLs often include an optic, and preferably at least one flexible fixation member or haptic which extends from the optic and becomes affixed in the eye to secure the lens in position. The optic normally includes an optically clear lens. Implantation of such IOLs into the eye involves making an incision in the eye. It is advantageous to have an incision size as small as possible to reduce trauma and speed healing.

Presently, apparatus for inserting intraocular lenses (IOLs) into eyes may be made of materials, in particular polymeric materials such as polypropylene, which have insufficient lubricity to facilitate the passage of a folded IOL through the inserter tube. One approach that may be considered is to use glycerol monostearate (GMS) as a lubricity agent in the hollow space of the tube to facilitate passing the IOL through the insertion apparatus. However, in an aqueous environment, GMS may dissolve in a pre-loaded IOL inserter where the inserter is stored in a saline buffer. Alternatively, when the lens placed in the IOL inserter glides over the surface of the inserter during insertion into the eye, the GMS may transfer onto the lens and end up in the eye, thereby creating the risk of causing trauma and/or irritation and/or damage to the eye which is undesirable.

Accordingly, it is desirable to provide improved apparatus for inserting IOLs into the eye such that the lens can be easily inserted into the eye.

### SUMMARY OF THE INVENTION

In accordance with one embodiment of the present invention, an apparatus for inserting an intraocular lens through a small incision into an eye is provided comprising a hollow tube including an interior wall defining a hollow space through which an intraocular lens may be passed and an outlet through which the intraocular lens may be passed from the hollow space into the eye, wherein at least the hollow tube of the apparatus is made from a polymeric resin comprising a polymer backbone and one or more pendent groups having peroxide functionality and covalently linked to the polymer backbone.

In accordance with a second embodiment of the present invention, a method for inserting an intraocular lens into an eye is provided comprising: (a) placing an outlet of a hollow tube in or in proximity to an incision in the eye, the hollow tube including an interior wall defining a hollow space containing an intraocular lens in a folded state, wherein at least the hollow tube of the of the apparatus is made from a polymeric resin comprising a polymer backbone and one or more pendent groups having peroxide functionality and covalently linked to the polymer backbone to facilitate passing the intraocular lens in the folded state through the hollow space; and (b) passing the intraocular lens from the hollow space through the outlet into the eye.

It is believed that by employing a polymeric resin comprising a polymer backbone and one or more pendent groups having peroxide functionality or a grafted polymeric product thereof in forming at least the hollow tube of an intraocular lens inserter, the hollow tube formed from the polymeric resin may be lubricious. Thus, an intraocular lens would be more easily released into the eye from the hollow tube of the intraocular lens inserter during surgery.

### BRIEF DESCRIPTION OF THE DRAWINGS

FIG. 1 is a front side view, in perspective, of an exemplary apparatus for inserting an IOL through a small incision into an eye in accordance with one embodiment of the present invention with the load chamber in the open position.

FIG. 2 is a side view, in perspective, of the exemplary apparatus shown in FIG. 1 with the load chamber in the closed position.

FIG. 3 is a front side view, in perspective, of the exemplary apparatus shown in FIG. 2 loaded into a hand piece.

FIG. 4 is a side view, partly in cross-section, taken generally along line 6-6 of FIG. 3.

FIG. 5 is a general schematic illustration showing the exemplary apparatus shown in FIG. 3, with the hand piece partially in cross-section, being used to insert an IOL into an eye.

### DETAILED DESCRIPTION OF THE PREFERRED EMBODIMENTS

One embodiment of the present invention is directed to an apparatus for inserting an intraocular lens through a small incision into an eye. In general, the apparatus will comprise a hollow tube including an interior wall defining a hollow space through which an intraocular lens may be passed and an outlet through which the intraocular lens may be passed from the hollow space into the eye, wherein at least the hollow tube of the apparatus is made from at least a polymeric resin comprising a polymer backbone and one or more pendent groups having peroxide functionality and covalently linked to the polymer backbone.

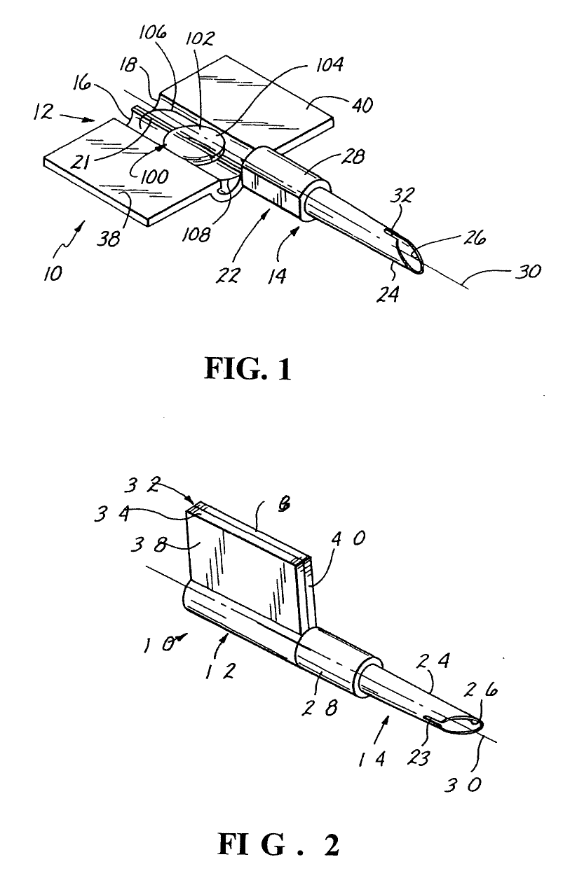

FIGS. 1 to 5 illustrate the use of IOL inserter 10 including exposed interior surfaces thereof formed from at least a polymeric resin comprising a polymer backbone and one or more pendent groups having peroxide functionality and covalently linked to the polymer backbone described hereinbelow. IOL inserter 10 is merely illustrative of the inserters included within the scope of the present invention. Inserters including the polymeric resin described herein can have configurations substantially different from IOL inserter 10 and are included within the scope of the present invention.

The body of IOL inserter 10 is an integrally formed, for example, molded, unit made of, for example, propropylene. Alternatively, the body of IOL inserter 10 can be made of the polymeric resin comprising a polymer backbone and one or more pendent groups having peroxide functionality and covalently linked to the polymer backbone or grafted polymeric product thereof, as described hereinbelow. For ease of manufacturing, it is preferable that the body of IOL inserter 10 be made of the same polymeric resin or grafted polymeric product thereof as the hollow tube of IOL inserter 10. Load chamber 12 includes a first member 16 and a second member 18 which are secured or joined together and are hingeably moveable relative to each other along line 21, which is parallel to the longitudinal axis 30 of inserter 10.

Injection tube 14 includes a proximal end portion 22, a distal end portion 24 and an open distal end 26. A reinforcing collar 28 is coincidental with the proximal end portion 22 of injection tube 14. Injection tube 14 also includes a through slot 32.

As shown in FIG. 1, inserter 10 is in the opened position. In contrast, in FIG. 2, inserter 10 is shown in the closed position. In the closed position, the load chamber 12 includes a top 32 which is a combination of top surfaces 34 and 36 of first wing 38 and second wing 40, respectively, of first member 16 and second member 18, respectively. First and second wings 38 and 40 are effective for a human user of inserter 10 to hold and manipulate the inserter 10 while using it, as described hereinbelow.

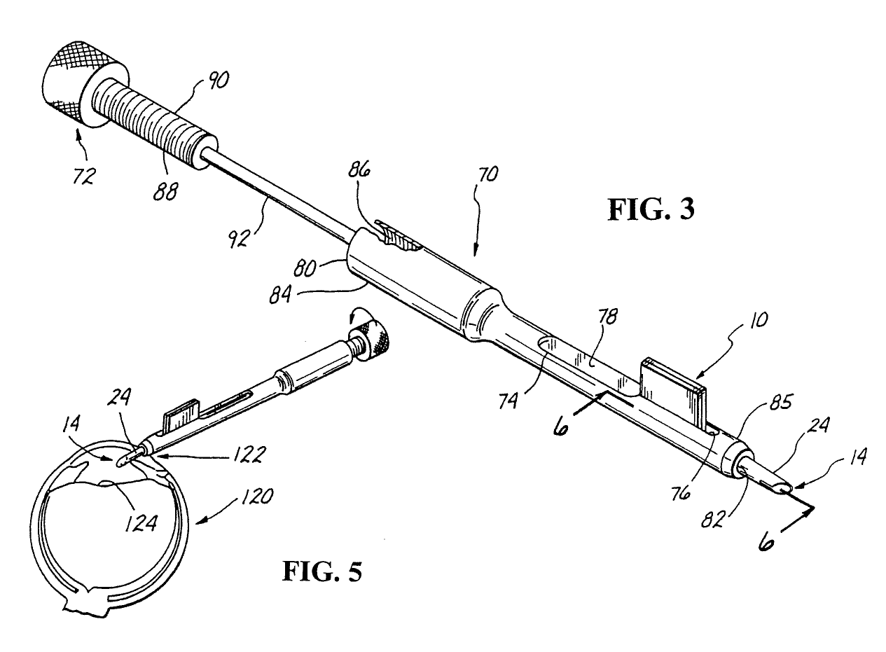

Inserter 10 is described in more detail with reference to FIG. 3, which shows the inserter in combination with hand piece 70. When used in combination with hand piece 70, the load chamber 12 of inserter 10 is in the closed position, as shown in FIG. 2.

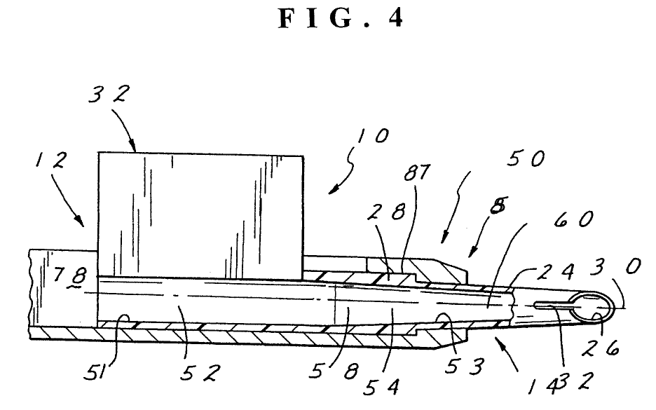

Referring to FIG. 4, with load chamber 12 in the closed position, the load chamber includes an interior wall 51 which defines a first lumen 52 that is elongated in a direction parallel to the longitudinal axis 30 of inserter 10. Injection tube 14 includes a tapering interior wall 53 which defines a distally tapering second lumen 54.

The first lumen 52 is aligned with the second lumen 54 so that a folded IOL in the first lumen can be passed directly from the first lumen into the second lumen. The taper of proximal portion 58 of second lumen 54 is more severe than the slight taper which exists in the distal portion 60 of the second lumen. The more severe taper in the proximal portion 58 is effective to further fold the IOL as the IOL is passed into the second lumen 54. This further folding is advantageous because the further folded IOL can be inserted into the eye through a smaller incision. The enhanced lubricity resulting from the polymeric resin comprising a polymer backbone and one or more pendent groups having peroxide functionality and covalently linked to the polymer backbone may facilitate this further folding so that a reduced amount of force is required to further fold the IOL and/or the degree of further holding of the IOL may be increased so that ultimately, the IOL can be inserted through an even smaller incision. The enhanced lubricity of the polymeric resin can also advantageously reduce the risk of tearing and/or otherwise damaging the IOL as the IOL is passed through the first lumen 52 and second lumen 54.

The polymeric resin for use in forming the hollow tube of the apparatus will include at least a polymer backbone and one or more pendent groups having peroxide functionality and covalently linked to the polymer backbone. In general, the polymeric material that forms the backbone of the polymeric resin can be a polyolefin. The polyolefin can be produced from one or more C₂ to C₂₀ alpha-olefin monomers. Representative examples of C₂ to C₂₀ alpha-olefin monomers include, but are not limited to, linear and branched alpha-olefins such as ethylene, propylene, 1-butene, 3-methyl-1-butene, 4-methyl-1-butene, 4-phenyl-1-butene, 1-pentene, 3-methyl-1-pentene, 4-methyl-1-pentene, 3,3-dimethyl-1-pentene, 3,4-dimethyl-1-pentene, 4,4-dimethyl-1-pentene, 1-hexene, 4-methyl-1-hexene, 5-methyl-1-hexene, 6-phenyl-1-hexene, 1-octene, 1-decene, 1-dodecene, 1-tetradecene, 1-hexadecene, 1-octadecene, 1-eicocene and the like and mixtures thereof; and halogen-substituted, linear and branched alpha-olefins such as hexafluoropropene, tetrafluoroethylene, 2-fluoropropene, fluoroethylene, 1,1-difluoroethylene, 3-fluoropropene, trifluoroethylene, 3,4-dichloro-1-butene and the like and mixtures thereof.

Although various polyolefins can be used herein, the preferred polyolefin that forms the backbone of the polymeric resin is polypropylene. The polypropylene homopolymers can have a weight average molecular weight ranging from about 200,000 to about 2,000,000. By way of example, the invention will be described herein with reference to the polymer backbone being a polypropylene backbone.

In general, the polymeric resin can be prepared by first subjecting the polypropylene material used as the backbone of the polymeric resin to a radical forming means. For example, the polymeric resin can be prepared by first exposing the polypropylene material to high energy ionizing radiation in an essentially oxygen-free environment, i.e., an environment in which the active oxygen concentration is established and maintained at, e.g., about 0.004% by volume or less, to form a polypropylene radical. The ionizing radiation should have sufficient energy to penetrate to the extent desired the mass of propylene polymer material being irradiated. The ionizing radiation can be of any kind, but the most practical kinds are electrons and gamma rays. Preferred are electrons beamed from an electron generator having an accelerating potential of about 500 to about 4000 kilovolts. Satisfactory results can be obtained at a dose of ionizing radiation of about 0.1 to about 15 megarads (“Mrad”), and preferably about 0.5 to about 9.0 Mrad.

The term “rad” is usually defined as that quantity of ionizing radiation that results in the absorption of 100 ergs of energy per gram of irradiated material, regardless of the source of radiation. Energy absorption from ionizing radiation is measured by the well known conventional dosimeter, a measuring device in which a strip of polymer film containing a radiation-sensitive dye is the energy absorption sensing means. Therefore, the term “rad” means that quantity of ionizing radiation resulting in the absorption of the equivalent of 100 ergs of energy per gram of the polymer film of a dosimeter placed at the surface of the propylene polymer material being irradiated.

The free radical-containing irradiated propylene polymer material is then subjected to an oxidative treatment step to provide a propylene polymer containing peroxy radicals (i.e., RCOO*). Generally, the oxidative treatment step involves heating the free radical-containing irradiated propylene polymer material in the presence of a controlled amount of active oxygen in the range of, for example, greater than about 0.004% but less than about 15% by volume, preferably less than about 8%, and most preferably less than about 3%, to a temperature of about 25° C. to about 140° C., more preferably about 40° C. to about 100° C., and most preferably about 50° C. to about 90° C. Heating to the desired temperature can be accomplished as quickly as possible, e.g., in less than about 10 minutes. The polymer is then held at the selected temperature, e.g., for about 5 to about 90 minutes, to increase the extent of reaction of the oxygen with the free radicals in the polymer. The holding time, which can easily be determined by one skilled in the art, will typically depend upon such factors as, for example, the properties of the starting material, the oxygen concentration used, the radiation dose, and the temperature. The maximum time is determined by the physical constraints of, for example, the fluid bed being used.

The oxidative treatment step can be carried out as one step, or the polymer can be heated in two steps, e.g., first at about 80° C. and then at about 140° C., while exposing the free radical-containing irradiated propylene polymer material to the specified amount of oxygen. For example, one way of carrying out the treatment in two steps is to pass the polypropylene radical through a first fluid bed assembly operating at T₁ in the presence of a controlled amount of oxygen, and then through a second fluid bed assembly operating at T₂ in the presence of a controlled amount of oxygen within the same range as in the first step.

The expression “active oxygen” means oxygen in a form that will react with the free radical-containing irradiated propylene polymer material. It includes molecular oxygen, which is the form of oxygen normally found in air. The active oxygen content requirement can be achieved by use of a vacuum or by replacing part or all of the air in the environment by an inert gas such as, for example, nitrogen or argon.

The concentration of peroxide groups formed on the polymer can easily be controlled by varying the radiation dose and the amount of oxygen to which the polymer is exposed after irradiation. The oxygen level in the fluid bed gas stream is controlled by the addition of air at the inlet to the fluid bed. Air must constantly be added to compensate for the oxygen consumed by the formation of peroxide groups on the polymer. The fluidizing medium can be, for example, nitrogen or any other gas that is inert with respect to the free radicals present, e.g., argon, krypton and helium.

Next, the propylene polymer containing peroxy radicals can undergo a hydrogen abstraction reaction as known in the art to provide peroxide species that are chemically bound to the propylene polymer backbone. Alternatively, the propylene polymer containing peroxy radicals can be reacted with a second polymer containing peroxy radicals to provide peroxide species that are chemically bound to the propylene polymer. The second polymer containing peroxy radicals can be prepared in the same manner as the first polymer containing peroxy radicals.

Finally, the propylene polymer containing peroxide species that are chemically bound to the propylene polymer are subjected to heat treatment to obtain a polymeric resin comprising a propylene polymer backbone and one or more pendent groups having peroxide functionality and covalently linked to the polymer backbone. Suitable temperatures for heat treatment can vary widely according to such factors as, for example, the specific propylene polymer used, and can range from about 50° C. to about 210° C. The reaction scheme for providing the polymeric resin comprising a propylene polymer backbone and one or more pendent groups having peroxide functionality and covalently linked to the polymer backbone is generally depicted in Scheme I below.

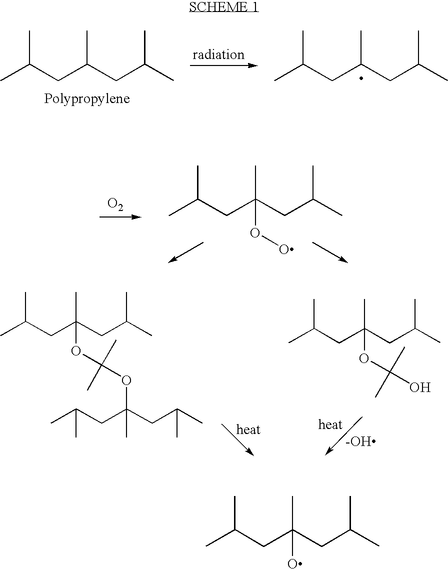

In one embodiment, one or more grafting monomers or polymers may then be grafted onto the polymeric resin comprising a propylene polymer backbone and one or more pendent groups having peroxide functionality and covalently linked to the polymer backbone. In general, the pendent groups having peroxide functionality in the propylene polymer backbone of the polymeric resin advantageously act as a source for free radicals. This, in turn, allows for the polymeric resin to react with an ethylenically unsaturated-containing radical to provide a graft polymeric product. Suitable grafting monomers and polymers that are capable of being grafted onto the polymeric resin include ethylenically unsaturated-containing radicals, such as, for example, unsaturated carboxylic acids, such as methacrylic and acrylic acids and the like; (meth)acrylic substituted alcohols, such as 2-hydroxyethylmethacrylate, 2-hydroxyethylacrylate, glyceryl methacrylate and the like; vinyl lactams, such as N-vinyl pyrrolidone and the like; (meth)acrylamides, such as methacrylamide, N,N-dimethylacrylamide and the like; vinyl alcohols, such as poly(vinyl alcohols) and the like; vinyl esters, such as vinyl acetate, poly(vinyl ester) polymers and the like; fluorinated polyolefin resins, such as polytetrafluoroethylene (Teflon®), polyvinylidenefluoride, tetrafluoroethylene/vinylidenefluoride copolymer, tetrafluoroethylene/hexafluoropropylene copolymer, ethylene/tetrafluoroethylene copolymer and the like; polyethylene polymers, such as high density polyethylene (HDPE), low density polyethylene (LDPE), linear low density polyethylene (LLDPE), very low density polyethylene (VLDPE), and the like, polystyrene (PS), and the like and combinations thereof. If desired, the vinyl ester moieties of the vinyl ester grafting monomers and polymers (e.g., poly(vinyl ester) polymer groups) of the grafted polymer resin can be saponified to vinyl alcohol moieties by reaction with an alkali such as sodium or potassium alkoxide thereby forming poly(vinyl alcohol) polymer groups.

Grafting of the foregoing grafting monomers and polymers onto the polymeric resin may be accomplished by methods known in the art. As used herein, the term “grafting” denotes covalent bonding of the grafting monomers or polymers to a polymer chain of the polymeric resin. The grafted polymeric products may be prepared in solution, in a fluidized bed reactor, or by melt grafting as desired. In one embodiment, a grafted polymeric product may be conveniently prepared under polymer melt reaction conditions by melt blending the ungrafted polymeric resin in the substantial absence of a solvent, and in the presence of the grafting monomers and/or polymers in a suitable reactor, e.g., in an extrusion reactor, a heated melt-blend reactor, a Banbury mill, etc.

In this embodiment, the polymeric resin will undergo heat treatment such that the peroxide functionalities on the propylene polymer backbone will advantageously act as a source of free radicals thereby reacting with the ethylenically unsaturated-containing grafting monomers and polymers. The graft polymerization reaction may be carried out at any suitable temperature. Suitable temperature ranges will depend on such factors as, for example, the desired level of grafting, the graft polymerization rate as a function of temperature for the monomer(s) employed, etc. For example, a suitable temperature can range from about 215° C. to about 350° C. However, one skilled in the art can readily determine suitable temperature ranges for a given grafting process.

To carry out the melt reaction, it is desirable to establish suitable reactor operating conditions for generating a grafted polymeric product having an effective percentage of or most or all of the grafting monomer and/or polymer grafted on the polymer. The grafting monomer and/or polymer should be grafted directly onto the polymeric resin, rather than forming dimeric, oligomeric, or homopolymeric graft moieties or, forming independent homopolymers.

One may generate a grafted polymeric product exhibiting the desired qualities and performance characteristics by selecting, for example, appropriate reactant feed rates as well as appropriate reactor operating conditions. These conditions include, among others, the proportions of the grafting monomer and polymer to the polymeric resin and as well as the design of the reactor and its operating conditions.

With reference to FIG. 3, inserter 10 is shown in combination with hand piece 70 and push rod member 72. Hand piece 70 includes a relatively large, elongated first through opening 74 and a relatively small, elongated second through opening 76. Hand piece 70 includes a through bore 78 which extends from the proximal end 80 to the distal end 82 of the hand piece. The proximal end portion 84 of hand piece 70 includes threads 86 which are adapted to engage and mate with threads 88 of the proximal segment 90 of push rod member 72. Rod element 92 of push rod member 72 is adapted to pass through bore 78, first lumen 52, second lumen 54 and out of open distal end 26. Hand piece 70 and push rod member 72 are made of metal, such as surgical grade stainless steel or the like metals. The distal end portion of rod member 72 can be made of a soft polymeric material, for example, configured to be introduced into and held in a fold of a folded IOL as the IOL is passed through the inserter.

Inserter 10 is operated and functions as follows. When it is desired to load an IOL into inserter 10, the inserter is placed, for example, manually placed, in a configuration as shown in FIG. 1. With load chamber 12 in the opened position, an IOL, such as one shown generally at 100, is placed, for example, using forceps, in between first and second members 16 and 18. This placement is such that the anterior face 102 of optic 104 faces upwardly, as shown in FIG. 1. If desired, it may be useful to employ a solution in the inserter to assist in preventing air bubbles. This solution may be a known viscoelastic solution or a balanced salt solution which is commonly used during eye surgery. The optic 104 can be made of a silicone polymeric material. The filament haptics 106 and 108 of IOL 100 are located as shown in FIG. 1, so that the fixation members are located generally parallel to, rather than transverse to, the longitudinal axis 30.

With IOL 100 placed as shown in FIG. 1, first and second members 16 and 18 are hingeably moved relative to each other, for example, by manually bringing first and second wings 38 and 40 together, to place the load chamber 12 in the closed position, as shown in FIG. 2. With load chamber 12 in the closed position, IOL 100 is in a folded state, that is optic 104 is folded. The relative movement of first and second members 16 and 18 to move the load chamber from the open position to the closed position is effective to fold the lens. The folded IOL 100 is now located in the first lumen 52. For clarity sake, the folded IOL is not shown in any of FIG. 2, 3, 4 or 5.

With the inserter 10 configured as shown in FIG. 2 and folded IOL 100 located in first lumen 52, the inserter 10 is placed in association with hand piece 70, as shown in FIG. 3. In this configuration, the distal end portion 24 of injection tube 14 extends distally beyond the distal end 82 of hand piece 70. As shown in FIG. 4, the distal portion 85 of hand piece 70 includes an inner wall 87 which is configured to receive reinforcing collar 28 in abutting relation.

With inserter 10 so placed relative to hand piece 70, push rod member 72 is pushed into the through bore 78 and into the inserter 10 to push the IOL 100 from the first lumen 52 into the second lumen 54. As the threads 88 come in contact with and engage threads 86, the push rod member 72 is rotated, as shown in FIG. 5, so as to thread the push rod member onto the proximal end portion 84 of hand piece 70. By gradually moving push rod element 92 through bore 78 of hand piece 70, the folded IOL 100 is urged to move from first lumen 52 into second lumen 54, through open distal end 26 and into the eye.

Referring now to FIG. 5, the IOL 100 is to be placed in eye 120 into an area formerly occupied by the natural lens of the eye. FIG. 5 shows the sclera 122 having an incision through which the distal end portion 24 of injection tube 14 is passed. Alternately, the incision can be made through the cornea. Distal end portion 24 has a sufficiently small cross-section to pass into the eye 120 through an incision in the sclera 122.

The injection tube 14 is manipulated within eye 122 until it is positioned so that IOL 100 can be properly positioned in eye 122, that is in the anterior chamber, the posterior chamber, the capsular bag 124 or in the sulcus, after being released. The surgeon is thus able to controllably position the distal end portion 24 of injection tube 14, with IOL 100 in the first lumen 52 of load chamber 12. Once distal end portion 24 is so positioned, the rod element 92 is urged distally, by rotating (threading) push rod member 72 onto hand piece 70, to pass the IOL 100 into and through the second lumen 54, through the open distal end 26 of injection tube 14 and into the eye 120. The anterior face 102 of IOL 100 faces generally forwardly in the eye 120 as the IOL is released from the inserter 10. In other words, the IOL 100 passes through first lumen 52, second lumen 54 and open distal end 26 and into eye 120 without flipping or otherwise becoming mispositioned. Only a relatively small amount of, if any, post-insertion re-positioning is needed to properly position IOL 100 in eye 120.

After the IOL 100 has been inserted into the eye, the rod element 92 is moved proximally into the injection tube 14 and the distal end portion 24 of the injection tube is removed from the eye. If needed, the IOL 100 can be repositioned in the eye by a small, bent needle or similar tool inserted into the same incision.

Once the IOL 100 is properly positioned in eye 120 and inserter 10 is withdrawn from the eye, the incision in the sclera may be mended, for example, using conventional techniques. After use, inserter 10 is preferably disposed of. Hand piece 70 and push rod member 72 can be reused, after sterilization/disinfection.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the features and advantages appended hereto.

What is claimed is:

1. An apparatus for inserting an intraocular lens through a small incision into an eye comprising a hollow tube including an interior wall defining a hollow space through which an intraocular lens may be passed and an outlet through which the intraocular lens may be passed from the hollow space into the eye, wherein at least the hollow tube of the apparatus is obtained from at least a polymeric resin comprising a polymer backbone and one or more pendent groups having peroxide functionality and covalently linked to the polymer backbone.

2. The apparatus of claim 1, wherein the polymer backbone of the polymeric resin comprises a polyolefin.

3. The apparatus of claim 2, wherein the polyolefin is polypropylene.

4. The apparatus of claim 1, wherein the polymer backbone of the polymeric resin comprises a polypropylene.

5. The apparatus of claim 1, wherein an ethylenically unsaturated-containing radical is grafted to the polymeric resin.

6. The apparatus of claim 5, wherein the ethylenically unsaturated-containing radical is selected from the group consisting of an unsaturated carboxylic acid, (meth)acrylic substituted alcohol, vinyl lactam, (meth)acrylamide, vinyl alcohol, vinyl ester, fluorinated polyolefin resin, polyethylene polymer and combinations thereof.

7. The apparatus of claim 6, wherein the unsaturated carboxylic acid comprises a methacrylic or acrylic-containing acid.

8. The apparatus of claim 6, wherein the (meth)acrylic substituted alcohol is selected from the group consisting of 2-hydroxyethylmethacrylate, 2-hydroxyethylacrylate, glyceryl methacrylate and combinations thereof.

9. The apparatus of claim 6, wherein the vinyl lactam is an N-vinyl pyrrolidone.

10. The apparatus of claim 6, wherein the (meth)acrylamide is selected from the group consisting of methacrylamide, N,N-dimethylacrylamide and combinations thereof.

11. The apparatus of claim 6, wherein the vinyl alcohol comprises a poly(vinyl alcohol).

12. The apparatus of claim 6, wherein the vinyl ester is vinyl acetate or a poly(vinyl ester) polymer.

13. The apparatus of claim 6, wherein the fluorinated polyolefin resin is a polytetrafluoroethylene resin.

14. The apparatus of claim 6, wherein the polyethylene polymer is selected from the group consisting of high density polyethylene (HDPE), low density polyethylene (LDPE), linear low density polyethylene (LLDPE), very low density polyethylene (VLDPE) and combinations thereof.

15. The apparatus of claim 1, wherein the hollow tube is obtained from a graft copolymer comprising the polymeric resin.

16. The apparatus of claim 1, further comprising a loading portion coupled to the hollow tube and sized and adapted to receive an intraocular lens for passage into the hollow space.

17. A method for inserting an intraocular lens into an eye, the method comprising:
(a) placing an outlet of a hollow tube in or in proximity to an incision in the eye, the hollow tube including an interior wall defining a hollow space containing an intraocular lens in a folded state, wherein the hollow tube is made from a polymeric resin comprising a polymer backbone and one or more pendent groups having peroxide functionality and covalently linked to the polymer backbone to facilitate passing the intraocular lens in the folded state through the hollow space; and
(b) passing the intraocular lens from the hollow space through the outlet into the eye.

18. The method of claim 17, wherein the polymer backbone of the polymeric resin comprises a polyolefin.

19. The method of claim 18, wherein the polyolefin is polypropylene.

20. The method of claim 17, wherein the polymer backbone of the polymeric resin comprises a polypropylene.

21. The method of claim 17, wherein an ethylenically unsaturated-containing radical is grafted to the polymeric resin.

22. The method of claim 21, wherein the ethylenically unsaturated-containing radical is selected from the group consisting of an unsaturated carboxylic acid, (meth)acrylic substituted alcohol, vinyl lactam, (meth)acrylamide, vinyl alcohol, vinyl ester, fluorinated polyolefin resin, polyethylene polymer and combinations thereof.

23. The method of claim 20, wherein an ethylenically unsaturated-containing radical is graft-copolymerized to the polymeric resin.
